(19) United States
(12) Patent Application Publication
(10) Pub. No.: US 2013/0197325 A1
(43) Pub. Date: Aug. 1, 2013

(54) ANTI-COAGULANT INFUSION FLUID SOURCE

(71) Applicants: James M. Petisce, Westford, MA (US); Jane M. Olin, Irvine, CA (US); Eden V. Paster, Irvine, CA (US); Kristie P. Trinh, Pico Rivera, CA (US)

(72) Inventors: James M. Petisce, Westford, MA (US); Jane M. Olin, Irvine, CA (US); Eden V. Paster, Irvine, CA (US); Kristie P. Trinh, Pico Rivera, CA (US)

(73) Assignee: Edwards Lifesciences Corporation, Irvine, CA (US)

(21) Appl. No.: 13/579,832

(22) PCT Filed: Mar. 1, 2011
(86) PCT No.: PCT/US11/26630

§ 371 Date: Jan. 4, 2013

Related U.S. Application Data
(60) Provisional application No. 61/310,236, filed on Mar. 3, 2010.

(51) Int. Cl. A61B 5/15; A61L 33/00

(52) U.S. Cl. CPC A61B 5/150992; A61L 33/0076
USPC 600/309; 427/2.12

(57) ABSTRACT

Methods and systems for preventing or eliminating blood coagulation or thrombus during use of an intravenous anti-thrombotic sensor are disclosed. The method further comprises for providing antimicrobial into the infusion fluid source. An anti-thrombotic sensor is also disclosed that comprises a coating of a complex of a non-heparin anti-thrombotic agent and an alkylbenzyldimethyl ammonium cationic salt.

### TECHNICAL FIELD

In general, embodiments herein disclosed relate to analyte measuring systems and, more specifically, methods and systems comprising an anticoagulant infusion fluid source for an analyte sensor and/or anticoagulant coatings for the analyte sensor.

### BACKGROUND

Controlling blood glucose levels for diabetics and other patients can be a vital component in critical care, particularly in an intensive care unit (ICU), operating room (OR), or emergency room (ER) setting where time and accuracy are essential. Presently, one of the most reliable ways to obtain a highly accurate blood glucose measurement from a patient is by a direct time-point method, which is an invasive method that involves drawing a blood sample and sending it off for laboratory analysis. This is a time-consuming method that is often incapable of producing needed results in a timely manner. Other minimally invasive methods such as subcutaneous methods involve the use of a lancet or pin to pierce the skin to obtain a small sample of blood, which is then smeared on a test strip and analyzed by a glucose meter. While these minimally invasive methods may be effective in determining trends in blood glucose concentration, they generally do not track glucose frequently enough to be practical for intensive insulin therapy, for example, where the impending onset of hypoglycemia could pose a very high risk to the patient.

Electrochemical sensors have been developed for measuring various analytes in an aqueous or physiological fluid mixture, such as the measurement of glucose in blood or serum. An analyte is a substance or chemical constituent that is determined in an analytical procedure, such as a titration. For instance, in an immunoassay, the analyte may be the ligand, antibody, DNA fragment, or other physiological marker, whereas in blood glucose testing the analyte is glucose. Electrochemical sensors comprise electrolytic cells including electrodes used to measure an analyte. Two types of electro-chemical sensors are potentiometric and amperometric sensors.

Amperometric sensors, for example, are known in the medical industry for analyzing blood chemistry. These types of sensors contain enzyme electrodes, which typically include an oxidase enzyme, such as glucose oxidase, that is immobilized within a membrane in proximity to the surface of an electrode. In the presence of blood, the membrane selectively passes an analyte of interest, e.g. glucose, to the oxidase enzyme, after which a byproduct of the enzymatic reaction is detected at the electrode. Amperometric sensors function by producing an electric current when a potential sufficient to sustain the reaction is applied between two electrodes in the presence of the reactants. For example, in the reaction of glucose and glucose oxidase, the hydrogen peroxide reaction product may be subsequently oxidized by electron transfer to an electrode. The resulting flow of electrical current in the electrode is indicative of the concentration of the analyte of interest in the media where the sensor is located.

Intravascular blood glucose (IVBG) sensor systems typically use an infusion fluid source containing a low level of heparin to prevent clotting in the tubing or in any dead-volume spaces of the sensor assembly used to sample blood for the glucose measurement from a patient. Prolonged exposure to heparin may lead to the formation of heparin induced thrombocytopenia (HIT).

### SUMMARY

The following presents a simplified summary of one or more embodiments in order to provide a basic understanding of such embodiments. This summary is not an extensive overview of all contemplated embodiments, and is intended to neither identify key or critical elements of all embodiments, nor delineate the scope of any or all embodiments. Its sole purpose is to present some concepts of one or more embodiments in a simplified form as a prelude to the more detailed description that is presented later.

In a first embodiment, a method for preventing or eliminating blood coagulation or thrombus during use of a sensor is provided. The method comprises providing an infusion fluid source, the infusion fluid source comprises a saline-based solution, an effective amount of at least one non-heparin, anti-thrombotic agent present in the saline-based solution, and providing an intravenous analyte sensor adapted for fluid communication with the infusion fluid source, where at least a portion of the analyte sensor is in contact with blood. The amount of at least one non-heparin, anti-thrombotic agent present in the saline-based is sufficient to prevent or eliminate blood coagulation or thrombus during use of the analyte sensor.

In a first aspect of the first embodiment, the at least one non-heparin, anti-thrombotic agent is salts of citric acid, dermatan sulfate, a complex of dermatan sulfate and a cationic alkylbenzyldimethyl ammonium salt; wherein the alkyl group is from 6 to 22 carbon atoms, Lepirudin, or Danaparoid.

In a second aspect, alone or in combination with one or more of the previous aspects of the first embodiment, the method further comprises an amount of at least one antimicrobial agent present in the saline-based solution sufficient to prevent or eliminate infection during use of the analyte sensor.

In a third aspect, alone or in combination with one or more of the previous aspects of the first embodiment, the at least one antimicrobial agent is taurolidine citrate.

In a fourth aspect, alone or in combination with one or more of the previous aspects of the first embodiment, the method further comprises providing a catheter adapted to house the analyte sensor, wherein at least one of the surfaces of the catheter is surface treated or surface coated to reduce or eliminate blood coagulation or thrombus.

In a fifth aspect, alone or in combination with one or more of the previous aspects of the first embodiment, the method further comprises providing a housing adapted to receive the analyte sensor.

In a sixth aspect, alone or in combination with one or more of the previous aspects of the first embodiment, the method further comprises providing a housing adapted to receive the analyte sensor, wherein at least one of the surfaces of the housing is surface treated or surface coated to reduce or eliminate blood coagulation or thrombus.

In a second embodiment a system for sensing an analyte of interest in a subject is provided. The system comprises an infusion fluid source comprising an amount of a non-heparin, anti-thrombotic agent present in saline-based solution sufficient to reduce or prevent blood coagulation or thrombus during use, and optionally, an amount of antimicrobial agent present in the saline-based solution sufficient to reduce or prevent infection during use; and an intravenous analyte sensor adapted for fluid communication with the infusion fluid source; and a controller electrically coupled to the sensor.

In a first aspect of the second embodiment, the at least one non-heparin, anti-thrombotic agent is dermatan sulfate, a citric acid salt, Lepirudin, or Danaparoid.

In a second aspect, alone or in combination with the previous aspect of the second embodiment, the at least one antimicrobial agent is taurolidine citrate.

In a third aspect, alone or in combination with one or more of the previous aspects of the second embodiment, the system further comprises a catheter adapted to house the sensor.

In a fourth aspect, alone or in combination with one or more of the previous aspects of the second embodiment, at least one of the surfaces of the catheter is surface treated or surface coated to reduce or eliminate blood coagulation or thrombus.

In a fifth aspect, alone or in combination with one or more of the previous aspects of the second embodiment, at least one of the surfaces of the catheter is contacted with a complex of dermatan sulfate and an alkylbenzyldimethyl ammonium salt, where the alkyl group is from 6 to 22 carbon atoms.

In a sixth aspect, alone or in combination with one or more of the previous aspects of the second embodiment, the system further comprising a housing adapted to receive the glucose sensor.

In an seventh aspect, alone or in combination with one or more of the previous aspects of the second embodiment, at least one of the surfaces of the housing is surface treated or surface coated to reduce or eliminate blood coagulation or thrombus.

In a eighth aspect, alone or in combination with one or more of the previous aspects of the second embodiment, at least one of the surfaces of the housing is contacted with a complex of dermatan sulfate and an alkylbenzyldimethyl ammonium salt, where the alkyl group is from 6 to 22 carbon atoms.

In a third embodiment an intravenous blood analyte sensor is provided. The analyte sensor comprises an intravenous analyte sensor having a surface configured for contacting blood and an anti-thrombogenic coating of a complex of dermatan sulphate and a cationic alkylbenzyldimethyl ammonium salt; wherein the alkyl group is from 6 to 22 carbon atoms; the coating contacting at least a portion of the surface of the analyte sensor.

In a first aspect of the third embodiment, the complex comprises stearylalkonium cation and dermatan sulfate.

In a second aspect, alone or in combination with any of the previous aspects of the third embodiment, the surface of the analyte sensor comprises a membrane comprising hydrophilic polymer and hydrophobic polymer.

In a fourth embodiment, a method for rendering an intravenous blood analyte sensor non-thrombogenic is provided. The method comprises providing an intravenous analyte sensor having at least one surface in contact with blood and contacting the at least one of the surfaces of the analyte sensor with a complex of dermatan and an alkylbenzyldimethyl ammonium cationic salt; wherein the alkyl group is from 6 to 22 carbon atoms.

In a first aspect, of the fourth embodiment, the coating step comprises providing a solution of the dermatan complex, applying the solution to the at least one surface of the analyte sensor, and drying the analyte sensor to form a coating thereon.

In a fifth embodiment, a method for reducing or eliminating heparin induced thrombocytopenia in a subject is provided. The method comprises providing an intravenous blood analyte sensor having at least one surface in contact with blood, and contacting the at least one of the surfaces of the analyte sensor with a complex of dermatan and an alkylbenzyldimethyl ammonium cationic salt; wherein the alkyl group is from 6 to 22 carbon atoms.

### BRIEF DESCRIPTION OF THE DRAWINGS

Having thus described embodiments of the invention in general terms, reference will now be made to the accompanying drawings, which are not necessarily drawn to scale, and wherein:

FIG. 1 is a schematic diagram of a system for blood glucose monitoring, according to an embodiment disclosed and described herein;

FIG. 2 is a flow diagram of a method for providing an infusion fluid source to a sensor, in accordance with aspects disclosed and described herein;

FIG. 3 is a flow diagram of a method for providing an infusion fluid source to a sensor, in accordance with aspects disclosed and described herein;

FIG. 4 is a flow diagram of a method for providing an infusion fluid source to a sensor, in accordance with aspects disclosed and described herein;

FIG. 5 is a flow diagram of a method for providing an infusion fluid source to a sensor, in accordance with aspects disclosed and described herein;

FIG. 6 is a flow diagram of a method for preventing or eliminating blood coagulation or thrombus by an intravenously positioned sensor, in accordance with aspects disclosed and described herein;

FIG. 7 is a flow diagram of a method for preventing or eliminating blood coagulation or thrombus by an intravenously positioned sensor, in accordance with aspects disclosed and described herein.

### DETAILED DESCRIPTION

Embodiments disclosed and described herein will now be described more fully hereinafter with reference to the accompanying drawings, in which some, but not all, embodiments disclosed and described herein are shown. Indeed, the spirit and scope of the claims can be embodied in many different forms and should not be construed as limited to the embodiments set forth herein; rather, these embodiments are provided so that this disclosure will satisfy applicable legal requirements. In the following description, for purposes of explanation, numerous specific details are set forth in order to provide a thorough understanding of one or more embodiments. It may be evident; however, that such embodiment(s) may be practiced without these specific details. Like numbers refer to like elements throughout.

Intravenous blood glucose sensor systems (IVBG) generally contain a sensor assembly that resides in proximity to a small catheter within a vein of the subject. To attain a blood glucose measurement, blood is accessed via the catheter and presented to the sensor. In some IVBG systems, after a glucose reading is obtained, a flush solution e.g., phosphate buffered saline and heparin is passed over the sensor from an IV bag connected to the system. Heparin is present to reduce or eliminate blood coagulation and/or thrombus formation. The IV bag may further contain a calibrant, for example a predetermined amount of glucose in order to calibrate the system.

Continue heparin exposure may cause heparin-induced-thrombocytopenia (HIT) in some subjects. HIT is essentially an immune response to an antigen formed by a complex of heparin and blood component PF4. HIT may induce a pro-coagulation state resulting in blood clots, which may form in the extremities such as the legs or arms, or in the heart (resulting in cardiac arrest) or in the brain (resulting in stroke). The probability of HIT occurrence has resulted in some hospitals banning heparin use completely, thus limiting the availability and benefits of IVBG systems.

Disclosed and described here is a method for reducing or eliminating anticoagulation during intravenous blood glucose sensing without the use of heparin. Method of preventing, reducing or eliminating heparin-induced-thrombocytopenia (HIT) are also provided.

In one aspect, what is disclosed and described is an IVBG system comprising a alkylbenzyldimethyl ammonium cationic salt of dermatan in contact with at least the glucose sensor component of IVBG sensor system. The term “dermatan,” as used herein, is inclusive of dermatan sulfate.

Dermatan sulfate is a glucosaminoglycan found in animal tissue. Dermatan sulfate is not a drug, but an endogenous naturally occurring substance. Dermatan sulfate is an effective anticoagulant in humans. Since it is generally believed that dermatan does not cause HIT, it can be used with patients predisposed to HIT, as well as all patients to reduce or eliminate the possibility of HIT.

The alkylbenzyldimethyl ammonium cationic salt of dermatan can be prepared by combining the dermatan with a alkylbenzyldimethyl ammonium cationic salt under conditions suitable for forming a complex. Suitable alkylbenzyldimethyl ammonium cationic salts include benzalkonium chloride (CAS RN: 8001-54-5) or benzethonium chloride (CAS RN: 121-54-0) or cetalkonium chloride (CAS RN: 122-18-9) or laurtrimonium bromide (CAS RN: 1119-94-4) or myristyltrimethylammonium bromide (CAS RN: 1119-97-7) or cetrimide (CAS RN: 8044-71-1) or cetrimonium bromide (CAS RN: 57-09-0) or cetylpyridinium chloride (CAS RN: 123-03-5) or stearalkonium chloride (CAS RN: 122-19-0). Mixtures of alkylbenzyldimethyl ammonium cationic salts may be used. In one preferred aspect, benzalkonium chloride is used to prepare the alkylbenzyldimethyl ammonium/dermatan complex. Commercially available benzalkonium chloride is believed to be a mixture of alkylbenzyldimethylammonium chlorides of the general formula, [C₆H₅CH₂N(CH₃)₂R]Cl, in which R represents a mixture of alkyls, including all or some of the groups comprising C₈ through C₂₂.

In one preferred aspect, a dermatan/quaternary ammonium complex is applied in proximity to the surface of the sensor of the IVBG system providing reduction or elimination of blood clots and/or thrombus. In one aspect, the dermatan/quaternary ammonium complex is applied to the outer membrane of the sensor of the IVBG system.

The alkylbenzyldimethyl ammonium cationic salts can be used in high loading concentrations with dermatan to form coatings having the above described beneficial features. Dermatan/quaternary ammonium complex can have at least 50 weight percent of the organic cationic salt and achieve coatings of acceptable quality. Weight percent as used herein means the ratios of the quaternary ammonium cation to the total weight of the complex. These weight percentages relate to, but are not limited by, the degrees of substitution of the cations on the dermatan molecule by the cationic quaternary ammonium salt.

In another aspect, an intravenous blood glucose (IVBG) sensor is provided. The IVBG sensor comprises a glucose sensor having a surface configured for contacting blood and an anti-thrombogenic surface coating of a complex of dermatan sulfate and a cationic alkylbenzyldimethyl ammonium salt, where the alkyl group is from 6 to 22 carbon atoms.

In a preferred aspect, the IVBG sensor surface configured for contacting blood comprises an anti-thrombogenic surface coating of a complex of stearylalkonium dermatan.

The IVBG sensor surface can comprise a membrane comprising hydrophilic polymer and hydrophobic polymer. For example, the membrane of the IVBG can be a silicone containing polycarbonate-polyurethane hydrophobic polymer and polyvinylpyrrolidone hydrophilic polymer. Other combinations of hydrophilic polymer and hydrophobic polymer may be used.

In another aspect, a method for rendering an intravenous blood glucose sensor (IVBG) non-thrombogenic is provided. The method comprises providing an intravenous blood glucose sensor (IVBG) having at least one surface in contact with blood and contacting the at least one of the surfaces of the IVBG sensor with a complex of dermatan and an alkylbenzyldimethyl ammonium cationic salt, where the alkyl group is from 6 to 22 carbon atoms. In one aspect, the method comprises providing an organic, aqueous or mixed organic/aqueous solution of the dermatan complex and contacting the solution to the at least one surface of the IVBG sensor and drying the IVBG sensor so as to form a coating thereon.

In other aspect, the method providing an intravenous blood glucose sensor (IVBG) having at least one surface in contact with blood and contacting the at least one of the surfaces of the IVBG sensor with a complex of dermatan and an alkylbenzyldimethyl ammonium cationic salt, where the alkyl group is from 6 to 22 carbon atoms is also envisaged as reducing or eliminating HIT.

In another aspect, the method comprises first contacting the IVBG sensor with an aqueous solution of a cationic quaternary ammonium organic salt, where the alkyl group is from 6 to 22 carbon atoms and subsequently contacting the IVBG sensor with an aqueous solution of dermatan salt.

In another aspect, methods and systems are defined for preparation of infusion fluid sources for an intravenous glucose sensor that does not contain heparin and prevents or eliminates blood clotting during blood sampling and measurement is provided. Thus, in one aspect, a method is disclosed and described comprising dermatan sulfate in the IV bag solution of the IVBG system in place of heparin for use in a hospital environment, and especially for use during surgical procedures or for diabetic patients. The method mitigates blood clotting and/or thrombus during use thereof and prevents or eliminates HIT.

In another embodiment, alone or in combination with the aspects disclosed above, a method for providing a premixed infusion fluid source is provided that includes saline-based solution, an anti-thrombotic agent, and an antimicrobial agent. In such embodiments, blood clotting, thrombus, and HIT problems as well as sensor-related infections are mitigated.

In an embodiment, a premixed infusion fluid source is provided that includes saline-based solution and an anti-thrombotic agent, optionally a buffer system comprising a predetermined concentration of at least one buffer. In such embodiments, in addition to addressing blood clotting problems, pH-related sensor deterioration during calibration and measurement sampling are also reduced or eliminated. Thus, an infusion fluid source, optionally comprising sufficient buffering capacity capable of providing a linear glucose verses current signal across a wide range of glucose values up to and including about 1000 mg/dL glucose is provided. This premixed infusion fluid source provides for accurate and consistent blood glucose concentration measurements during use of an intravenous glucose sensor.

It is generally believed that by providing a buffering capacity in an infusion fluid source, the signal of a glucose sensor is stabilized to an extent greater than that of a similar sensor exposed to an un-buffered infusion fluid source. While not to held to any particular theory, it is believed that the buffered infusion fluid source prevents or eliminates buildup of acidic byproduct and prevents or eliminates an acidic pH shift in and around the sensor environment by rapidly neutralizing the acidic by-products. For example, in an enzymatic glucose sensor, the gluconic acid formed in the glucose oxidase (GOx) catalyzed oxidation of glucose may be effectively neutralized, or the local environmental pH may be maintained near a predetermined value or range.

According to a first embodiment, an infusion fluid source with an anti-coagulant, such as citrate or citric acid/citrate that comprises a quantity of either phosphate or bicarbonate, either present in higher than physiological or normal concentrations but the resultant fluid having a similar osmolality to human blood, such that a stable glucose signal is provided. Citrate concentration may be between 0.5-4% wt/v % (0.019 M-0.15 M). Citric acid/citrate solutions of between about 1:2 and 1:20 molar ratio (citric/citrate) may be used. Citrate may be used for providing both anti-thrombotic/anticoagulation function as well as buffering. Citrate may be the anti-thrombotic agent/anticoagulant and the sole component of the buffering system.

Phosphate concentration may be between about 0.020 M and about 0.120 M. Phosphate and citrate buffering systems may be comprised of between about 0.020 M and about 0.120 M phosphate and between about 0.019 M and about 0.15 M citrate.

Bicarbonate concentration may be between about 20 mM and about 100 mM such as to provide a physiological pH. Bicarbonate and citrate buffering systems may be comprised of between about 20 mM and about 100 mM bicarbonate and between about 0.019 M and about 0.15 M citrate. As used herein, “bicarbonate” or “bicarbonate ion” is inclusive of carbonate ions and the mixture of bicarbonate and carbonate ions normally or abnormally present in biological fluids.

Phosphate/bicarbonate/citrate buffering systems concentrations may be comprised of between about 0.020 M and about 0.120 M phosphate, between about 20 mM and about 100 mM bicarbonate, and between about 0.019 M and about 0.15 M citrate. Such buffering systems can be provided in the above specified ranges provided the osmolality of the solution is not excessive (e.g., about 320 mOsm+/−10%). Sodium, potassium, and ammonium salts of citrate, bicarbonate, or phosphate may be used.

According to an aspect of the first embodiment, the infusion fluid source provides buffering capacity to an implanted intravenous blood glucose sensor such that a physiological mammalian pH range, or a pH range between a pH of about 6.50 and about 7.6, is provided.

According to one aspect of the first embodiments, the infusion fluid source comprises an anti-thrombotic agent to prevent and/or eliminate blood coagulation or thrombus (blood clotting) in the sensor assembly during use. Anti-thrombotic agents include, for example, anti-platelet agents, thrombolytic agents, and non-heparin anticoagulants such as direct thrombin inhibitors. Suitable anti-platelet agents include P2Y12 receptor inhibitors. Suitable anti-platelet agents include thienopyridine compounds, for example, Clopidogrel, (marketed under the tradename Plavix, Clopilet, or Ceruvin), ticlopidine or prasugrel. Suitable anti-platelet agents include platelet aggregation inhibitors. Suitable thrombolytic agents include, for example, vitamin K antagonists, tissue plasminogen activators (t-PA), Alteplase (Activase), reteplase (Retavase), tenecteplase (TNKase), Anistreplase (Eminase), streptokinase (Kabikinase, Streptase), and urokinase (Abbokinase). Suitable non-heparin anticoagulants include, for example, univalent direct throbin inhibitors such as Argatroban, Dabigatran, Melagatran, and Ximelagatran, or bivalent direct throbin inhibitors such as Hirudin, Bivalirudin (Angiomax), Lepirudin, and Desirudin. Other thrombotic agents may be used, such as Dabigatran, Defibrotide, Dermatan sulfate, Fondaparinux (Arixtra), citrate, sodium citrate, citric acid/citrate, and Rivaroxaban (Xarelto). Combinations of thrombotic agents as listed above may be used. A combination of heparan sulfate, dermatan sulfate and chondroitin sulfate (Danaparoid) may also be used.

In one aspect, taurolidine citrate (TCS) is used as an antimicrobial and anti-thrombotic. TCS prevents the formation of biofilms in the catheter lumen, which is especially advantageous in diabetic patients that are prone to infections at least in part because of their hyperglycemia. Also, because TCS is an amino acid derivative, it is likely nontoxic for Total Parenteral Nutrition (TPN) administration, dialysis, and long term indwelling catheters. In one aspect, TCS citrate is used alone in the infusion fluid source.

In another aspect of the first embodiment, the infusion fluid source comprises an antimicrobial agent to prevent and/or eliminate infections in the human patient during use of the sensor assembly. Suitable antimicrobial agents include, for example, taurolidine citrate. Other antimicrobials may be used, for example, one or more antivirals, antibiotics, antifungals, antiparasitics, acetic acid, essential oils, or silver and its salts.

In another aspect of the first embodiment, the method provides for the infusion fluid source further including providing the infusion fluid source that includes the saline-based solution, optionally a predetermined concentration of calibrant, such as glucose, a non-heparin based anti-thrombotic agent, and an antimicrobial agent.

In a second embodiment, a system comprising an infusion fluid source comprising an anti-thrombotic agent and an antimicrobial agent in combination with an intravenous glucose sensor is provided. The system includes an infusion fluid source including a saline-based solution, an anti-thrombotic agent, and an antimicrobial agent. The system additionally includes a sensor.

According to specific embodiments of the system, the system additionally includes a housing adapted to receive the sensor. In one aspect, surfaces of the housing are treated or are coated to reduce or eliminate blood coagulation or thrombus.

The term “calibrant” as used herein is inclusive of one or more analytes of interest believed to be present in the environment of the sensor during use, and exogenous compounds or compositions of matter that may be used to calibrate a sensor. In a particularly preferred embodiment, the calibrant is glucose, glucose in combination with one or more analytes of interest other than glucose, exogenous compounds or compositions of matter that may be used to calibrate a sensor, or combinations thereof.

The method herein disclosed provides a convenient manner for use in a hospital environment. In one aspect, discussed in detail infra, a premixed infusion fluid source is provided that includes saline-based solution and a predetermined concentration of an anti-thombotic agent or anticoagulant together with an antimicrobial agent.

Embodiments disclosed and described herein will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. In the following description, for purposes of explanation, numerous specific details are set forth in order to provide a thorough understanding of one or more embodiments. It may be evident; however, that such embodiment(s) may be practiced without these specific details. Like numbers refer to like elements throughout.

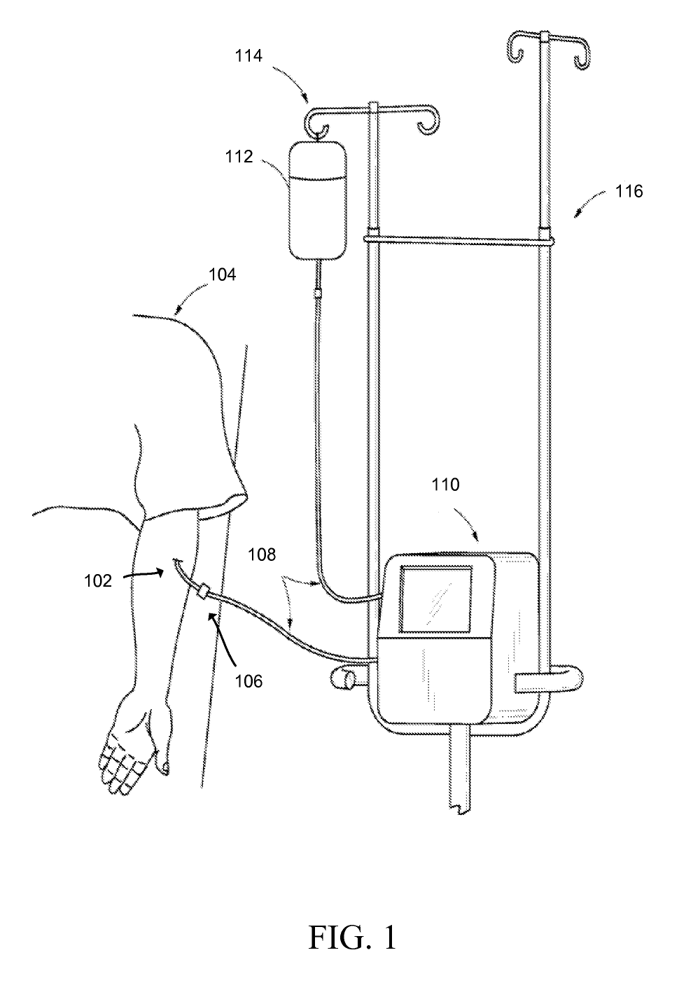

In one aspect disclosed and described herein, the intravenous blood glucose (IVBG) sensor system illustrated in FIG. 1 is employed. The phrase “glucose sensor” is inclusive of additional analyte sensors or sensors in addition to the glucose sensor. System 100 of FIG. 1 includes a sensor assembly 102, for example, as described in United States Patent Application Publication No.: 2008/00860427, which is incorporated herein by reference, that is intravenously inserted to a patient 104. The sensor assembly 102 is connected to the patient via an intravenous (IV) housing 106 and an infusion line 108, which is operably connected to a fluid controller (not shown) that is controlled by a control unit 110. The housing and/or catheter may be surface treated or surface coated to prevent or eliminate blood coagulation or thrombus. Finally, the infusion line 108 continues upstream of the fluid controller to an infusion fluid source 112, such as an infusion fluid bag, which may be supported by member 114. The system may be attached to a support structure 116. In one embodiment, member 114 may serve as a scale (piezoelectric or spring) operable to weigh the bag and send the weight to the controller.

During calibration mode of system 100, control unit 110 controls and meters infusion fluid from the infusion fluid source 112, past sensor assembly 102, and into the patient 104. The sensor assemblies preferably include sensing electrodes constructed, for example, as described in U.S. Patent Application Publication Nos.: 2009/0143658, 2009/0024015, 2008/0029390, 20070202672, 2007/0202562, and 2007/0200254, which are incorporated herein by reference, and during calibration, the current generated by the respective electrodes of the sensor (e.g., a working electrode and a blank electrode) assembly is measured to provide calibration measurements for system 100.

During measurement mode of the system, blood is urged past the sensor by reversing the fluid controller. In one aspect, blood may be prevented from being withdrawn from the patient 104. In another aspect, blood from the patient may be drawn past sensor assembly 102 but preferably not past control unit 110. While blood is in contact with the sensor assembly the current or other detectable signal generated by the respective electrodes is measured.

In one embodiment, substantially the same flow rates are used during calibration mode and during measurement mode. More particularly, the control system controls the infusion of the system such that the infusion fluid is urged past the sensor electrodes at a fixed flow rate during calibration, and the blood measurement is taken while the blood is drawn back from the patient at approximately the same flow rate. Other flow rates for the calibration and measurement modes may be used.

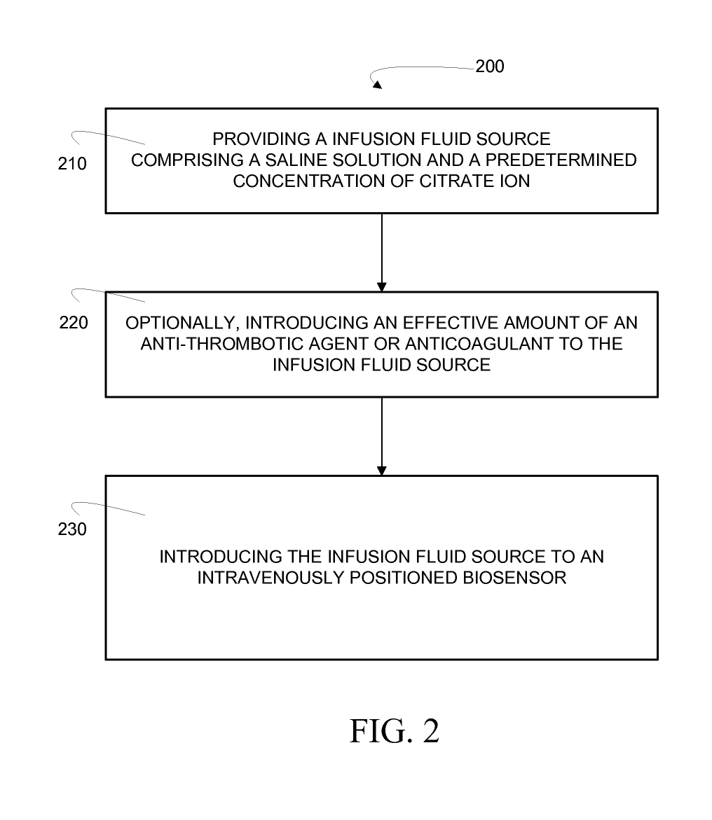

Referring to FIG. 2, a flow diagram is presented of a method 200 for preparing an infusion fluid source, in accordance with embodiments disclosed and described herein. At Event 210, a predetermined concentration of citrate ion is introduced to an infusion fluid source that includes saline-based solution.

At Event 220, an effective amount of an anti-thrombotic agent and/or anticoagulant is optionally introduced to the infusion source. The introduction of the citrate ion and the optional anti-thrombotic agent and/or anticoagulant may be carried out in any order or may be introduced simultaneously.

At Event 230, the infusion source comprising the citrate ion is introduced to an intravenously positioned sensor, e.g. a glucose sensor, thereby insuring the accuracy of the resulting concentration of glucose determined by the sensor.

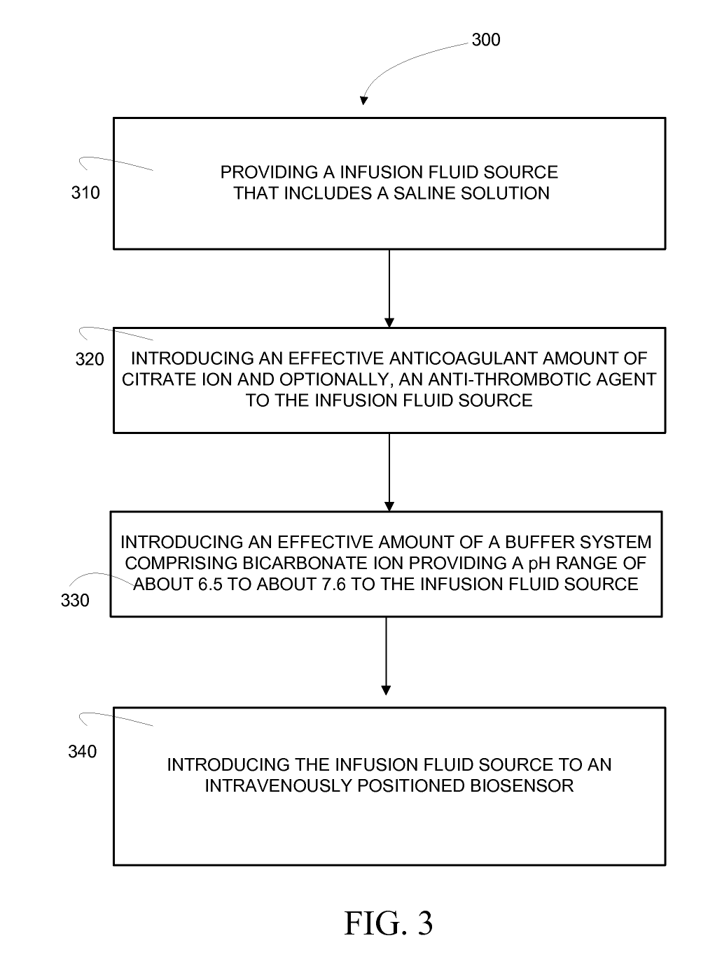

Referring to FIG. 3 a flow diagram is presented of an alternate method 300 for preparing an infusion fluid source in accordance with embodiments disclosed and described herein comprising a source of citrate ion in combination with a bicarbonate buffer. At Event 310, an infusion fluid source is provided that includes a saline-based solution.

At Event 320, an effective anticoagulant amount of citrate ion and optionally an anti-thrombotic agent is introduced to the infusion source. The introduction of the citrate ion and the optional anti-thrombotic agent may be carried out in any order or may be introduced simultaneously.

At Event 330, an effective amount of a buffer system comprising bicarbonate ion is introduced to the infusion source to provide a pH range of about 6.5 to about 7.6. The introduction of the bicarbonate buffer and citrate ion may be carried out in any order or may be introduced simultaneously provided that a pH range of about 6.5 to about 7.6 is targeted.

At Event 340, the infusion source comprising the effective amount of buffer system comprising bicarbonate ion and the effective amount of citrate ion is introduced to an intravenously positioned sensor, e.g. a glucose sensor, thereby insuring the accuracy of the resulting concentration of glucose determined by the sensor.

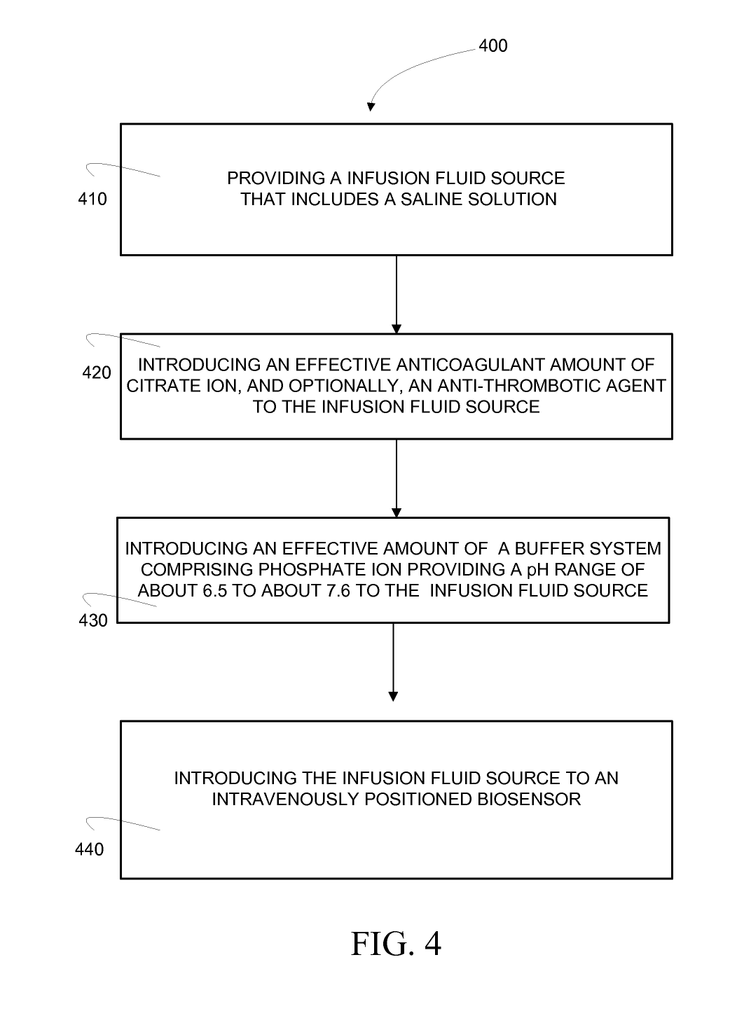

Referring to FIG. 4, a flow diagram is presented of an alternate method 400 for preparing an infusion fluid source in accordance with embodiments disclosed and described herein comprising a source of citrate ion in combination with a bicarbonate buffer. At Event 410, an infusion fluid source is provided that includes a saline-based solution.

At Event 420, an effective anticoagulant amount of citrate ion and optionally an anti-thrombotic agent is introduced to the infusion source. The introduction of the citrate ion and the optional anti-thrombotic agent may be carried out in any order or may be introduced simultaneously.

At Event 430, an effective amount of a buffer system comprising phosphate is introduced to the infusion source to provide a pH range of about 6.5 to about 7.6. The introduction of the phosphate buffer and citrate ion may be carried out in any order or may be introduced simultaneously provided that a pH range of about 6.5 to about 7.6 is provided.

At Event 440, the infusion source comprising the effective amount of buffer system comprising phosphate, the effective amount of citrate ion, and optional anti-thrombotic is introduced to an intravenously positioned sensor, e.g. a glucose sensor, thereby insuring the accuracy of the resulting concentration of glucose determined by the sensor.

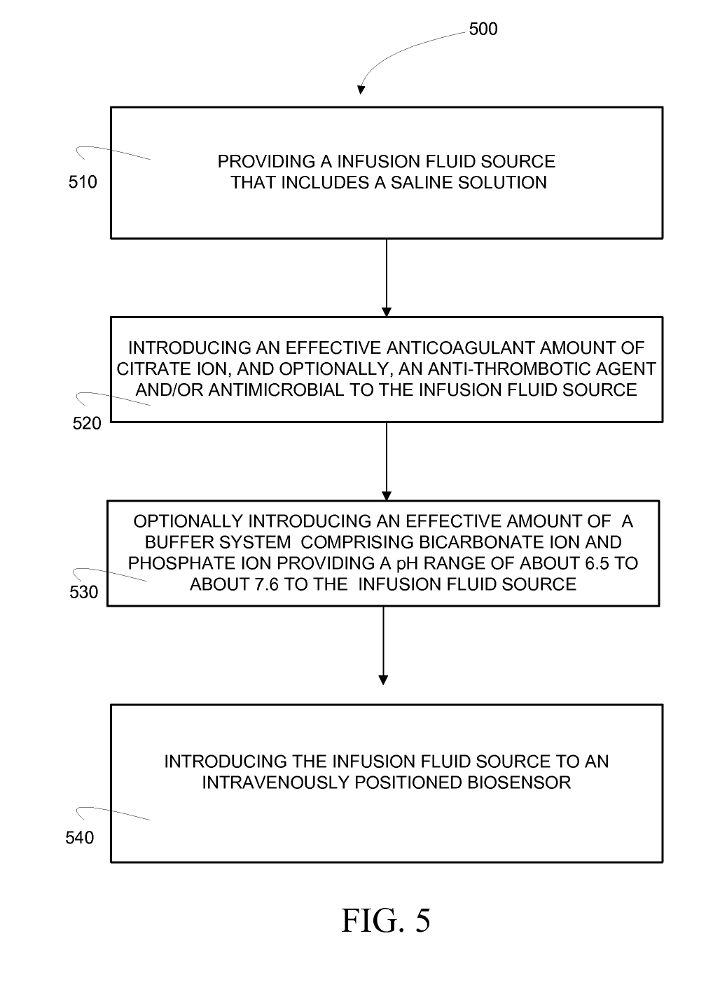

Referring to FIG. 5, a flow diagram is presented of an alternate method 500 for preparing an infusion fluid source in accordance with embodiments disclosed and described herein comprising a source of citrate ion in combination with a bicarbonate buffer. At Event 510, an infusion fluid source is provided that includes a saline-based solution.

At Event 520, an effective anticoagulant amount of citrate ion and optionally an anti-thrombotic agent and/or antimicrobial is introduced to the infusion source. The introduction of the citrate ion and the optional anti-thrombotic agent may be carried out in any order or may be introduced simultaneously.

At Event 530, optionally, an effective amount of a buffer system comprising bicarbonate ion and phosphate is introduced to the infusion source to provide a pH range of about 6.5 to about 7.6. The introduction of the bicarbonate/phosphate buffer, citrate ion and optional anti-thrombotic agent may be carried out in any order or may be introduced simultaneously provided that a pH range of about 6.5 to about 7.6 is provided.

At Event 540, the infusion source comprising the effective amount of buffer system comprising bicarbonate/phosphate, the effective amount of citrate ion and optional anti-thrombotic agent is introduced to an intravenously positioned sensor, e.g. a glucose sensor, thereby insuring the accuracy of the resulting concentration of glucose determined by the sensor.

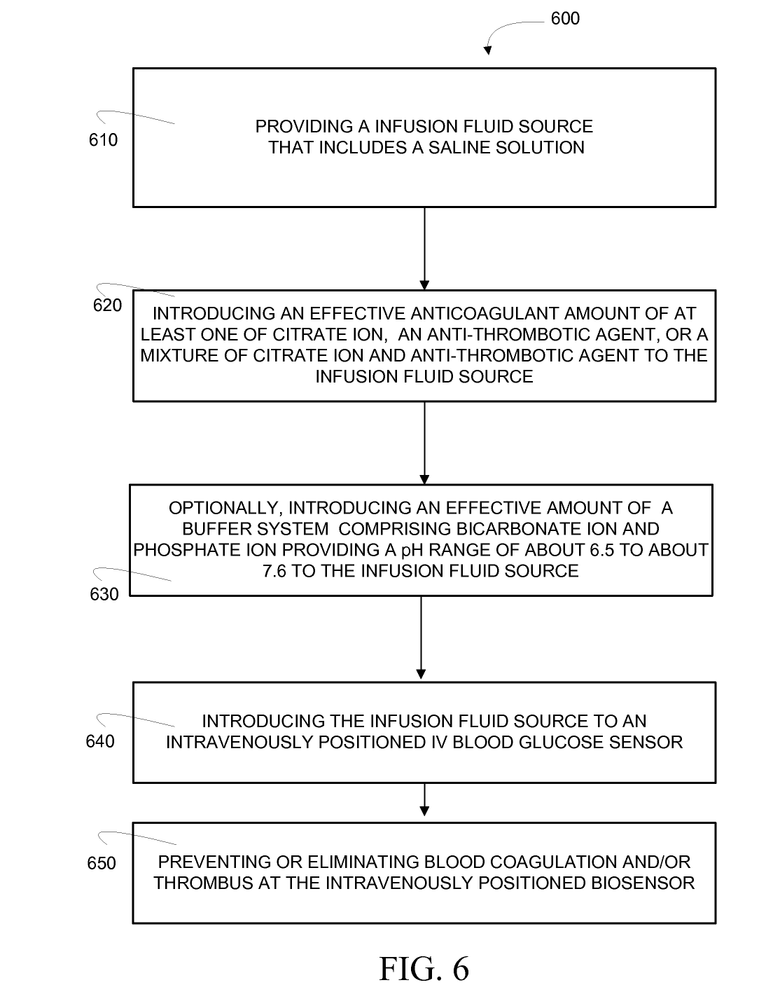

Referring to FIG. 6, a flow diagram is presented of a method 600 for preventing or eliminating thrombus in the intravenously positioned IV sensor, e.g., intravenous blood glucose sensor. At Event 610, an infusion fluid source is provided that includes a saline-based solution.

At Event 620, an effective anticoagulant amount of at least one of citrate ion, anti-thrombotic agent, or a mixture of citrate ion and anti-thrombotic agent is introduced to the infusion source. The introduction of citrate ion and/or the anti-thrombotic agent may be carried out in any order or may be introduced simultaneously.

At Event 630, an effective amount of a buffer system comprising bicarbonate ion and phosphate is introduced to the infusion source to provide a pH range of about 6.5 to about 7.6. The introduction of the effective amount of citrate or anti-thrombotic agent and buffer system may be carried out in any order or may be introduced simultaneously provided that a pH range of about 6.5 to about 7.6 is provided.

At Event 640, the infusion source comprising the effective amount of buffer system and the effective anticoagulant amount of citrate or anti-thrombotic agent is introduced to an intravenously positioned sensor, e.g. a glucose sensor, preventing or eliminating thrombus therein.

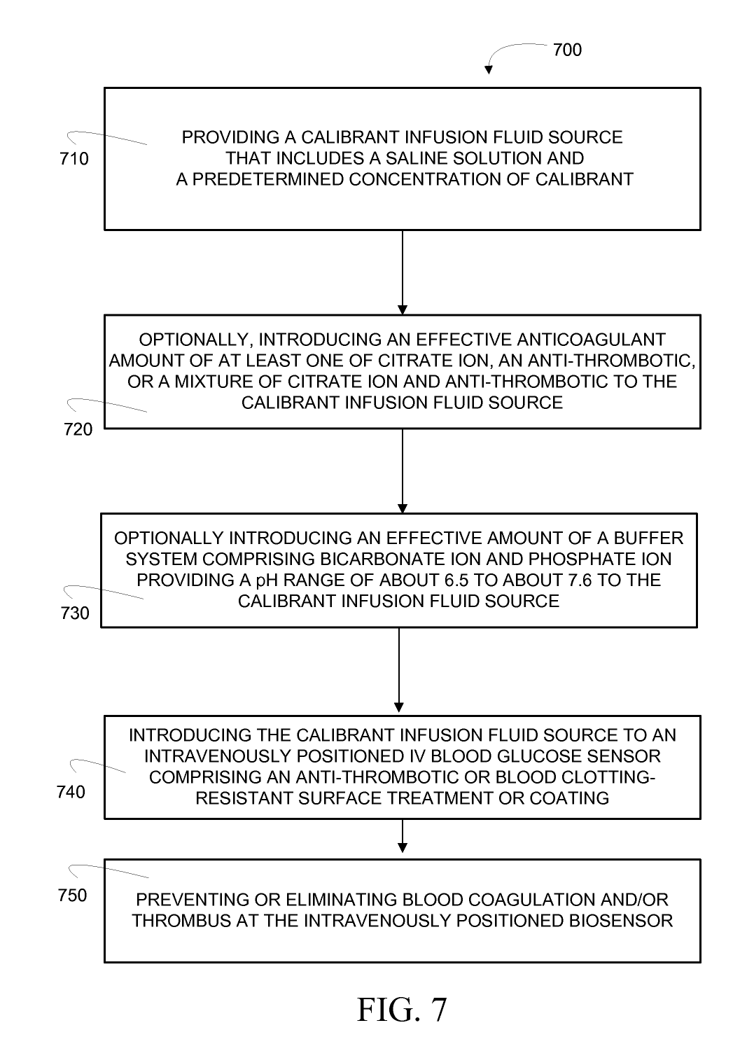

Referring to FIG. 7, a flow diagram is presented of a method 700 for preventing or eliminating thrombus in the intravenously positioned IV sensor, e.g., intravenous blood glucose sensor. At Event 710, an infusion fluid source is provided that includes a saline-based solution.

At optional Event 720, an effective anticoagulant amount of at least one of citrate ion and/or an anti-thrombotic agent is introduced to the infusion source. The introduction of citrate and/or the anti-thrombotic agent may be carried out in any order or may be introduced simultaneously.

At optional Event 730, an effective amount of a buffer system comprising bicarbonate ion and phosphate is introduced to the infusion source to provide a pH range of about 6.5 to about 7.6. The introduction of the effective amount of citrate and/or the anti-thrombotic agent and buffer system may be carried out in any order or may be introduced simultaneously provided that a pH range of about 6.5 to about 7.6 is provided.

At Event 740, the infusion source comprising the optional effective amount of citrate and/or the anti-thrombotic agent and the optional effective amount of buffer system is introduced to an intravenously positioned sensor e.g. a glucose sensor, comprising an anti-thrombotic surface coating as further described and disclosed herein. Any of the surfaces that may come into contact with blood can be surface treated or surface coated to reduce or eliminate blood coagulation or thrombus, such as tubing, catheter, sensor substrate, housing, or combinations thereof. For example, one or more of the surfaces of the catheter is contacted with an alkylbenzyldimethyl ammonium salt of dermatan sulfate. At Event 750, the anti-thrombic surface coated intravenously positioned sensor prevents or eliminates thrombus therein.

### Surface Treatments and Coatings

Various methods may be used, alone or in combination with the infusion fluid source described above, for providing a material with a modified surface resistant to blood coagulation or thrombus and/or having anti-thrombotic properties. For example, a sensor housing or support (e.g., catheter) may be physically coated, or chemically bonded to an alkylbenzyldimethyl ammonium salt and then coupled, with an anti-thrombotic agent. This can be done by incorporating an amine in the polymer comprising the housing or support, quaternizing the amine, and then coupling the anti-thrombotic agent to the quaternized material to provide an ionically bound anti-thrombotic agent. A benzalkonium salt of an anti-thrombotic agent such as dermatan sulfate, for example, can be used to treat or coat a sensor housing or support such as a catheter. In one aspect, stearylalkonium salt is preferred to reduce or prevent saline wash-off of the anti-thrombotic agent, for example, during the calibration step or during flushing. Various chemical surface modifications of the sensor or support can be used to anchor the agent, for example, gas-discharge plasma methods, corona discharge surface activation, e-beam or gamma surface activation. In other aspects, a complex of a stearylalkonium salt and dermatan in an alcoholic solvent is used to coat the surface of any one or more of the housing, the catheter, and the sensor.

In another aspect, use of a complex of dermatan and an alkylbenzyldimethyl ammonium cationic salt, wherein the alkyl group is from 6 to 22 carbon atoms, for the manufacture of an intravenous blood analyte sensor for reducing or preventing heparin induced thrombocytopenia in a subject during use of the analyte sensor is provided.

In another aspect, use of a complex of dermatan and an alkylbenzyldimethyl ammonium cationic salt; wherein the alkyl group is from 6 to 22 carbon atoms, for the manufacture of an intravenous blood analyte sensor for reducing or preventing blood clotting and/or thrombus in a subject during use of the analyte sensor.

### EXAMPLES

### Example 1

Taurolidine citrate: Subjects in need of an IVBG system can be administered taurolidine citrate in an amount of between about 0.1% to about 5% taurolidine citrate in an amount between about 1% to about 7% via IV infusion (weight/volume). The pH may be adjusted with citric acid and/or sodium hydroxide. In a more preferred aspect, subjects in need of an IVBG system can be administered taurolidine citrate in an amount of about 1.35% taurolidine citrate via infusion. It is believed that in subjects having an IVBG infusion fluid source comprising taurolidine citrate elimination or reduction of catheter related sepsis would result in combination with reduced or eliminated blood clotting or thrombus, without affecting the performance of the blood glucose sensor.

### Example 2

Dermatan Sulphate: Subjects in need of an IVBG system can be administered dermatan sulphate as an IV bolus injection followed by IV drip in an amount of between about 0.01% to about 0.04%. In a more preferred aspect, subjects in need of an IVBG system can be administered dermatan sulphate in an amount of about 0.03% via infusion. It is believed that in subjects having an IVBG infusion fluid source comprising dermatan sulphate, elimination or reduction of blood clotting or thrombus would result, without affecting the performance of the blood glucose sensor.

### Example 2

Preparation of alkylbenzyl ammonium cation-dermatan complex: 27 grams of dermatan sulfate (Celsus, Inc., Cincinnati, Ohio) was dissolved in 215 milliliters of distilled water. The solution was mixed with a 420 milliliter of a water solution containing 63 grams of purified benzalkonium chloride (Sigma Aldrich, St. Louis, Mo.). This complex compound was separated from solution by means of filtration. The alkylbenzyl ammonium cation-dermatan complex was dissolved in isopropanol for coating of the intravenous sensor. Coating was performed by dipping the sensor in the isopropanol solution for a few seconds and allowing the coating to dry in ambient air for a few minutes. Other coating methods may be used, such as brush coating, spraying, or vapor deposition.

Thus, present embodiments provide for methods and systems for preparation and use of infusion fluid sources for intravenously positioned sensors. This method also provides a sensor capable of preventing or eliminating blood coagulation or thrombus for use in a hospital environment. In another embodiment, a sensor capable of preventing or eliminating infections is provided.

While the foregoing disclosure discusses illustrative embodiments, it should be noted that various changes and modifications could be made herein without departing from the scope of the described aspects and/or embodiments as defined by the appended claims. Furthermore, although elements of the described aspects and/or embodiments may be described or claimed in the singular, the plural is contemplated unless limitation to the singular is explicitly stated. Additionally, all or a portion of any embodiment may be utilized with all or a portion of any other embodiment, unless stated otherwise.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A method for preventing or eliminating blood coagulation or thrombus during use of an intravenous sensor, the method comprising:
providing an infusion fluid source, the infusion fluid source comprising:
a saline-based solution;
an amount of at least one non-heparin, anti-thrombotic agent present in the saline-based solution; and
providing an intravenous analyte sensor adapted for fluid communication with the infusion fluid source, wherein at least a portion of the analyte sensor is in contact with blood;
wherein the amount of at least one non-heparin, anti-thrombotic agent present in the saline-based solution is sufficient to prevent or eliminate blood coagulation or thrombus during use of the analyte sensor.

2. A method of claim 1, wherein the at least one non-heparin, anti-thrombotic agent is a complex of dermatan salt and an alkylbenzyldimethyl ammonium salt; wherein the alkyl group is from 6 to 22 carbon atoms.

3. A method of claim 1, wherein the at least one non-heparin, anti-thrombotic agent is a complex of dermatan sulphate and an alkylbenzyldimethyl ammonium salt selected from at least one of stearylalkonium chloride, myristylalkonium chloride, eicosanylalkonium chloride, and docosylalkonium chloride.

4. A method of claim 1, wherein the at least one non-heparin, anti-thrombotic agent at least one of a salt of citric acid, taurolidone citrate, dermatan sulfate, Lepirudin, or Danaparoid.

5. A method of any one of claims 1-4, further comprising an amount of at least one antimicrobial agent present in the saline-based solution sufficient to prevent or eliminate infection during use of the analyte sensor.

6. A method of claim 5, wherein the at least one antimicrobial agent is taurolidine citrate.

7. A method of any one of claim 1-4 or 6, further comprising providing a catheter adapted to house the analyte sensor, wherein at least one of the surfaces of the catheter and/or sensor is surface treated or surface coated to reduce or eliminate blood coagulation or thrombus.

8. A method of any one of claim 1-4 or 6, further comprising providing a housing adapted to receive the analyte sensor, the housing adapted for fluidic coupling with a catheter, tubing, and the infusion source.

9. A method of claim 8, wherein at least one of the surfaces of the housing or tubing is surface treated or surface coated to reduce or eliminate blood coagulation or thrombus.

10. A system for sensing an analyte of interest in a subject, the system comprising:
an infusion fluid source comprising an amount of a non-heparin, anti-thrombotic agent present in saline-based solution sufficient to reduce or prevent blood coagulation or thrombus during use, and optionally, an amount of antimicrobial agent present in the saline-based solution sufficient to reduce or prevent infection during use; and
an intravenous analyte sensor adapted for fluid communication with the infusion fluid source; and
a controller electrically coupled to the sensor.

11. A system of claim 10, wherein the at least one non-heparin, anti-thrombotic agent is dermatan sulfate, a salt of citric acid, Lepirudin, or Danaparoid.

12. A system of claim 10, wherein the at least one antimicrobial agent is taurolidine citrate.

13. A system of any one of the claims 10-12, further comprising a catheter configured to house the sensor.

14. A system of any one of the claims 10-12, further comprising a coating associated with the analyte sensor, the coating comprising a complex of dermatan sulfate and a cationic an alkylbenzyldimethyl ammonium salt; wherein the alkyl group is from 6 to 22 carbon atoms.

15. A system of claim 14, wherein the complex comprises dermatan sulfate and an alkylbenzyldimethyl ammonium salt selected from at least one of stearylalkonium chloride, myristylalkonium chloride, eicosanylalkonium chloride, and docosylalkonium chloride.

16. A system of any one of the claims 10-12, wherein at least one of the surfaces of the catheter is surface treated or surface coated to reduce or prevent blood coagulation or thrombus.

17. A system of any one of the claims 10-12, further comprising a housing, the housing coupled to the catheter or adapted to receive at least a portion of the sensor, and tubing fluidically coupling the catheter with the infusion source.

18. A system of claim 17, wherein at least one of the surfaces of the housing or tubing is contacted with a complex of dermatan sulphate and a cationic an alkylbenzyldimethyl ammonium salt; wherein the alkyl group is from 6 to 22 carbon atoms.

19. An intravenous blood analyte sensor comprising:
an intravenous analyte sensor having a surface configured for contacting blood; and
an anti-thrombogenic coating of a complex of dermatan sulphate and a cationic an alkylbenzyldimethyl ammonium salt; wherein the alkyl group is from 6 to 22 carbon atoms; the coating contacting at least a portion of the surface of the analyte sensor.

20. An intravenous blood analyte sensor of claim 19, wherein the complex comprises dermatan sulfate and a an alkylbenzyldimethyl ammonium salt selected from at least one of stearylalkonium chloride, myristylalkonium chloride, eicosanylalkonium chloride, and docosylalkonium chloride.

21. An intravenous blood analyte sensor of any one of claims 19-20, wherein the surface comprises a membrane comprising hydrophilic polymer and hydrophobic polymer.

22. A method for rendering an intravenous blood analyte sensor non-thrombogenic, the method comprising:
providing an intravenous analyte sensor having at least one surface in contact with blood; and
contacting the at least one of the surfaces of the analyte sensor with a complex of dermatan and an alkylbenzyldimethyl ammonium salt; wherein the alkyl group is from 6 to 22 carbon atoms.

23. A method for reducing or eliminating heparin induced thrombocytopenia in a subject, the method comprising:
providing an intravenous blood analyte sensor having at least one surface in contact with blood; and
contacting the at least one of the surfaces of the analyte sensor with a complex of dermatan and an alkylbenzyldimethyl ammonium salt; wherein the alkyl group is from 6 to 22 carbon atoms.

24. A method for reducing or eliminating heparin induced thrombocytopenia in a subject, the method comprising:
providing an intravenous blood analyte sensor having at least one surface in contact with blood; and
contacting the at least one of the surfaces of the analyte sensor with a complex of dermatan and an alkylbenzyldimethyl ammonium salt; wherein the alkyl group is from 6 to 22 carbon atoms.

25. A method for reducing or eliminating sepsis in a subject, the method comprising:
providing a saline-based infusion solution comprising an antimicrobial;
providing an intravenous analyte sensor having at least one surface configured for contacting blood, the analyte sensor adapted for fluid communication with the saline-based infusion solution, wherein least one of the surfaces of the analyte sensor comprises a non-heparin anti-thrombotic coating.

26. The method of claim 25, wherein the complex of non-heparin anti-thrombotic coating dermatan and an alkylbenzyldimethyl ammonium salt; wherein the alkyl group is from 6 to 22 carbon atoms.

27. An intravenous blood analyte sensor system comprising:
a saline-based infusion solution comprising an antimicrobial agent;
an intravenous analyte sensor having a surface configured for contacting blood, the analyte sensor adapted for fluid communication with the saline-based infusion solution; and
an anti-thrombogenic coating comprising a complex of dermatan sulphate and an alkylbenzyldimethyl ammonium salt; wherein the alkyl group is from 6 to 22 carbon atoms, the coating contacting at least a portion of the surface of the analyte sensor.

28. Use of a complex of dermatan and an alkylbenzyldimethyl ammonium salt, wherein the alkyl group is from 6 to 22 carbon atoms, for the manufacture of an intravenous blood analyte sensor for reducing or preventing heparin induced thrombocytopenia in a subject during use of the analyte sensor.

29. Use of a complex of dermatan and an alkylbenzyldimethyl ammonium salt; wherein the alkyl group is from 6 to 22 carbon atoms, for the manufacture of an intravenous blood analyte sensor for reducing or preventing blood clotting and/or thrombus in a subject during use of the analyte sensor.
